(19) United States
(12) Patent Application Publication
(10) Pub. No.: US 2023/0160878 A1
(43) Pub. Date: May 25, 2023

(54) METHOD OF SCREENING A THERAPY FOR INDUCTION OF FERROPTOSIS

(71) Applicant: ARPEGGIO BIOSCIENCES, INC., Boulder, CO (US)

(72) Inventors: Timothy READ, Boulder, CO (US); Joseph AZOFEIFA, Boulder, CO (US); Maria LAI, Boulder, CO (US); Joel BASKEN, Boulder, CO (US)

(21) Appl. No.: 18/150,753

(22) Filed: Jan. 5, 2023

Related U.S. Application Data
(63) Continuation of application No. PCT/US2021/040542, filed on Jul. 6, 2021.
(60) Provisional application No. 63/048,253, filed on Jul. 6, 2020.
(60) Provisional application No. 63/074,980, filed on Sep. 4, 2020.

(51) Int. Cl. C12Q 1/26

(52) U.S. Cl. CPC C12Q 1/26; C12Q 2600/136; G01N 2333/902

(57) ABSTRACT

Provided herein are methods of screening a therapy for induction of ferroptosis, the method comprising: exposing a cell line to a potential therapy; measuring induction of Heme Oxygenase 1 (HMOX1) resulting from the exposing the cell line to the potential therapy; and identifying the potential therapy as passing or failing the screening of the therapy for induction of ferroptosis based on the measuring the induction of HMOX1.

### 1. CROSS-REFERENCE TO RELATED APPLICATIONS

This application is a continuation application of International Application No. PCT/US2021/040542, filed Jul. 6, 2021, which claims the benefit of priority of U.S. provisional patent application No. 63/048,253, filed on Jul. 6, 2020, and U.S. provisional patent application No. 63/074,980, filed on Sep. 4, 2020, the disclosures of which are incorporated herein by this reference in their entireties.

### 2. FIELD OF THE INVENTION

The invention relates to the field of functionally linking genetic loci, screening for biological markers and in certain aspects to screening biological markers for potential therapies.

### 3. BACKGROUND OF THE INVENTION

Most disease-associated genetic variants exist within non-protein coding regions of the genome, making their functional interpretation difficult (Zhu, Y., Tazearslan C., and Y. Suh, Experimental Biology and Medicine ((2017) 242(13): 1325-1334, which is incorporated herein by reference in its entirety). Many of these variants function by altering transcriptional regulation of distal genes, resulting in aberrant gene expression patterns and disease phenotypes. Single nucleotide polymorphisms (SNPs) identified in Genome Wide Association Studies (GWAS) are enriched within enhancer elements, i.e., regions of DNA critical for proper regulation of distal gene transcription. Because enhancers can regulate multiple genes from great genomic distances, linking enhancers (including those harboring disease associated variants) to their target genes is challenging.

There have been several large-scale attempts to link enhancers to target genes, including consortiums such as GeneHancer (Fishilevich, S., et al., Database (2017) 2017: bax028, which is incorporated herein by reference in its entirety) and Enhancer Atlas (Gao. T, and J. Qian, Nucleic Acids Research (2020) 48(D1): D58-D64, which is incorporated herein by reference in its entirety). Such approaches typically integrate multiple “-omics” data types across cell types and/or conditions and identify correlations between molecular features present at enhancers and genes. For example, the presence of chromatin mark H3K27Ac can be used to identify active enhancers by chromatin immunoprecipitation sequencing (ChIP-seq), albeit non-quantitatively (Creyghton, M. P., et al., Proceedings of the National Academy of Sciences (2010) 107(50): 21931-21936, which is incorporated herein by reference in its entirety). However, to make correlations between the presence of an active enhancer and the expression level of a gene, ChIP-seq data needs to be combined with RNA sequencing data. RNA sequencing methods like PRO-seq (Mahat, D. G., et al., Nature Protocols (2016) 11(8): 1455-1476, which is incorporated herein by reference in its entirety) and Cap Analysis of Gene Expression (CAGE) (Shiraki, T., et al., Proceedings of the National Academy of Sciences (2003) 100(26): 15776-15781, which is incorporated herein by reference in its entirety) are able to detect transcription from both enhancers and genes, however, these methods are difficult and time-consuming, preventing high degrees of reproducibility and throughput.

Connections between disease-relevant enhancers (i.e., enhancers harboring disease-associated SNPs) and their dysregulated gene targets can provide valuable insight into the molecular mechanisms of a disease process and suggest potential avenues for diagnostic opportunities or therapeutic intervention. There is a need in the art for methods of linking disease-associated enhancer variants to the genes they regulate that are sensitive and dynamic, cost effective, and scalable.

### 4. SUMMARY OF THE INVENTION

The invention provides a method of functionally linking two or more genetic loci. The method may include exposing a cell sample to a plurality of different stimuli in parallel for a period of time sufficient to elicit a transcriptional response directly or indirectly; performing a transcriptional run-on assay on the exposed cell samples where nascent RNA transcripts are labeled and isolating cellular RNA from the cell samples; enriching the labeled nascent RNA transcripts and preparing a library; sequencing the nascent RNA library and mapping it to a reference genome, wherein individual mapped sequencing reads represent genomic locations where active transcription was occurring at the end of the stimulus period; optionally, performing one or more quality control protocols on the sequence data; and identifying active enhancers throughout the genome and correlating enhancer activity with gene expression across the cell samples.

In certain embodiments, the stimuli comprise any or all of: an epigenetic modulator, a reader of histone modifications, a writer of histone modifications, an eraser of histone modifications, a DNA methyltransferase, a DNA methylase, a modulator of a cell signaling pathway, a pathway inhibitor, a modulator of cancer etiology, a MAPK, a JAK/STAT, a NFKB, a transcription factor, a p53, an ER, an AR, a GR, a MYC, a component of the proteasomal degradation system, or a deubiquitinating enzyme, and combinations thereof. In certain embodiments, exposing the cell sample may include in vitro exposure, in vivo exposure, or in vivo exposure in an animal model. In certain embodiments, the period of time may include about 60 minutes or less, about 30 minutes or less, about 15 minutes or less, or about 5 minutes or less. In certain embodiments, the period of time may include a series of exposure times. The series of exposure times may for example be about 5 minutes, about 15 minutes, about 1 hour, about 6 hours, or about 24 hours. The series of exposure times may for example be about every 5 minutes, about every 15 minutes, about every 1 hour, about every 6 hours, or about every 24 hours.

In certain embodiments, the cell samples may include one or more of: animal cells, human cells, a cell line, a xenograft model, a blood sample, cancer cells, cancer cells with known mutations, cancer cells representing multiple cancer stages, Phase I-, Phase II-, Phase III-, and/or Phase IV-cancer cells, hematopoietic cells, stem cells, progenitor cells, mature cells of the hematopoietic system, T cells, a T cell line, neuronal cells, or neuronal cells representative of a neurodegenerative disease and/or process. In certain embodiments, the cell sample may include two or more different cell samples. In certain embodiments, the cell sample may include two or more different types of cells.

In certain embodiments, the quality control protocol may include ensuring that the enrichment was for nascent RNA over steady state RNA by calculating an exon-intron ratio and identifying a minimum number of enhancers and promoters. In certain embodiments, the correlating enhancer activity with gene expression may include use of a machine learning-based model.

The method may include integrating a separate dataset to determine how non-coding variants or enhancer variants affect distal gene transcription and/or disease processes. For example, the second data set may include one or more of the following: single nucleotide polymorphisms (SNPs) identified by Genome Wide Association Studies (GWAS); mutations discovered by sequencing cancer cells relative to healthy cells from a patient; rare disease enhancer-linked mutations discovered by whole genome sequencing of an affected individual and the parents of the affect individual relative to the general population; epigenomic DNA sequencing data from the cell sample being analyzed; cell-free DNA or RNA data from a bodily fluid from the subject whose cells are being analyzed in the cell sample; Hi-C data for the cell sample being analyzed providing a measurement of physical proximity of two genomic loci; Hi-ChIP data for the cell sample being analyzed providing a measurement of physical proximity of proteins associated with DNA; ATAC-seq for the cell sample being analyzed providing a measurement of regions of “open,” or accessible chromatin; and/or ChIP-seq for the cell sample being analyzed providing a measurement of transcription factor occupancy or histone modifications.

The invention provides a method of identifying a marker for a biological interaction. In one embodiment, the includes: quantifying genome-wide RNA expression in cells absent a perturbagen; exposing the cells to a perturbagen selected to induce the biological interaction, thereby inducing changes in RNA expression in the cells; quantifying genome-wide nascent RNA expression in the cells exposed to the perturbagen; quantifying the difference between: nascent RNA expression in the cells absent the perturbagen; nascent RNA expression in the cells exposed to the perturbagen; and identifying as a marker any expressed RNA for which the difference quantified in step (d) is a statistical outlier relative to other expressed RNA.

In certain embodiments, the perturbagen causes a known biological interaction. In certain embodiments, exposing the cells to the perturbagen may include in vitro exposure, in vivo exposure, or in vivo exposure in an animal model. In certain embodiments, exposing the cells to the perturbagen may include a period of time of about 60 minutes or less, about 30 minutes or less, about 15 minutes or less, or about 5 minutes or less. In certain embodiments, exposing the cells to the perturbagen may include a series of exposure times. The series of exposure times may for example, be about 5 minutes, about 15 minutes, about 1 hour, about 6 hours, or about 24 hours. The series of exposure times may for example, be about 5 every minutes, about every 15 minutes, about every 1 hour, about every 6 hours, or about every 24 hours.

In certain embodiments, the marker may include a single RNA. In certain embodiments, the marker may include two or more different RNAs. In certain embodiments, the marker may include two or more sets of RNAs. In certain embodiments, the marker may include an enhancer RNA.

In certain embodiments, the cells may include one or more of animal cells, human cells, a cell line, a xenograft model, a blood sample, cancer cells, cancer cells with known mutations, cancer cells representing multiple cancer stages, Phase I-, Phase II-, Phase III-, and/or Phase IV-cancer cells, hematopoietic cells, stem cells, progenitor cells, mature cells of the hematopoietic system, T cells, a T cell line, neuronal cells, or neuronal cells representative of a neurodegenerative disease and/or process. In certain embodiments, the cells comprise a ferroptosis-sensitive cancer cell line. In certain embodiments, the cells comprise two or more different cell lines. In certain embodiments, the cells comprise two or more different cell types.

In certain embodiments, the perturbagen may include two or more perturbagens selected to induce the biological interaction.

In certain embodiments, the statistical outlier may include an expressed RNA at least 1.5 standard deviations from the nearest RNA. In certain embodiments, the statistical outlier may include an expressed RNA at least 2 standard deviations from the nearest RNA. In certain embodiments, the statistical outlier may include an expressed RNA at least 2.5 standard deviations from the nearest RNA. In certain embodiments, the statistical outlier may include an expressed RNA at least 3 standard deviations from the nearest RNA. In certain embodiments, the statistical outlier may include an expressed RNA at least 4 standard deviations from the nearest RNA. In certain embodiments, the statistical outlier may include an expressed RNA at least 5 standard deviations from the nearest RNA.

The method of may include quantifying a dose dependent response of the cells to the perturbagen.

The invention provides a method of screening a therapy for induction of ferroptosis. In one embodiment, the method may include: exposing a cell line to a potential therapy; measuring induction of HMOX1 resulting from the exposing; and identifying the potential therapy as passing or failing the screening test based on the measured induction of HMOX1.

In certain embodiments, the cell line may include a ferroptosis-sensitive cell line. The ferroptosis-sensitive cell line may, for example, be a cancer cell line. In certain embodiments, the cell line may include a ferroptosis-resistant cell line.

In certain embodiments, the cell line may include one or more of animal cells, human cells, a xenograft model, a blood sample, cancer cells, cancer cells with known mutations, cancer cells representing multiple cancer stages, Phase I-, Phase II-, Phase III-, and/or Phase IV-cancer cells, hematopoietic cells, stem cells, progenitor cells, mature cells of the hematopoietic system, T cells, a T cell line, epithelial cells, skin cells, esophageal cells, colorectal cells, neuronal cells, or neuronal cells representative of a neurodegenerative disease and/or process.

In certain embodiments, the therapy may include a drug therapy. In certain embodiments, the therapy may include a potential inhibitor of GPX4. The potential inhibitor of GPX4 may include a small molecule, a protein, or a peptide. The potential inhibitor of GPX4 may include using a gene therapy technique to modulate GPX4 function.

The gene therapy technique may include a gene editing technique.

Measuring induction of HMOX1 may include quantifying the level of HMOX1 gene expression.

Quantifying the level of HMOX1 may, for example, include measuring: an increase in HMOX1 gene expression; no change in HMOX1 gene expression; or a reduction in HMOX1 gene expression. In certain embodiments, measuring induction of HMOX1 may include measuring a change in HMOX1 mRNA abundance. In certain embodiments, measuring induction of HMOX1 may include quantifying the level of HMOX1 gene transcription using a nascent transcription assay. In certain embodiments, measuring induction of HMOX1 may include measuring protein expression. In certain embodiments, measuring induction of HMOX1 may include using RT-qPCR to quantify HMOX1 expression.

In certain embodiments, the potential therapy is identified as passing the screening test if the measured induction is increased expression of HMOX1 relative to a control. In certain embodiments, the potential therapy is identified as passing the screening test if the measured induction is an increased expression of HMOX1 of at least 1.5× relative to the control. In certain embodiments, the potential therapy is identified as passing the screening test if the measured induction is an increased expression of HMOX1 of at least 2× relative to the control. In certain embodiments, the potential therapy is identified as passing the screening test if the measured induction is a lack of change or a reduced expression of HMOX1 relative to the control.

The method may include generating a score for the potential therapy based on degree of induction of HMOX1. Generating the score may be performed without directly quantifying accumulation of lipid peroxides.

The method may include measuring cell death in response to exposure to the potential therapy. Measuring cell death may, for example, include using a live/dead assay. The live/dead assay may, for example, include a two-color fluorescence-based assay, wherein one color is used to detect live cells and a second color is used to detect dead cells. The degree of cell death may be used in combination with the degree of the measured induction of HMOX1 to score the potential therapy.

In certain embodiments, the cell line may include a ferroptosis-sensitive cell line, the method further comprising: exposing the ferroptosis-sensitive cell line to the potential therapy in the presence of a lipophilic antioxidant; measuring induction of HMOX1 resulting from the exposing in the presence of the lipophilic antioxidant; and comparing induction of HMOX1 on exposure to the potential therapy in the absence of the lipophilic antioxidant and in the presence of the lipophilic antioxidant, wherein induction of HMOX1 in the absence but not in the presence of the lipophilic antioxidant indicates ferroptosis without generalized oxidative stress and induction of HMOX1 in both the presence and the absence of the lipophilic antioxidant indicates ferroptosis with generalized oxidative stress. For example, a potential therapy that induces HMOX1 in the absence but not in the presence of the lipophilic antioxidant may be indicated to pass the screen. The lipophilic antioxidant may, for example, include a ferrostatin. The ferrostatin may include, for example, ferrostatin-1 or an active modified version of ferrostatin-1 or combinations thereof. The lipophilic antioxidant may include liproxstatin. The lipophilic antioxidant may include an iron chelator. The method may include measuring cell death.

A potential therapy identified as passing the screening test may manufactured in a therapeutically acceptable form. A potential therapy identified as passing the screening test may be administered to a subject in need thereof in a therapeutically effective amount to treat a disease condition. The therapy may induce expression of HMOX1 in the absence but not in the presence of the lipophilic antioxidant.

### 5. BRIEF DESCRIPTION OF FIGURES

FIG. 1 is a flow diagram illustrating an example of a workflow of the invention. In this case, the workflow is for simultaneously capturing changes in transcriptional activity at enhancers and genes throughout the genome of a cell to define the regulatory landscape of the cell in response to a perturbagen.

FIG. 2 is a schematic diagram illustrating a correlation matrix for linking transcriptional activity between enhancers and distal genes using nascent RNA sequence data.

FIG. 3A is a plot showing a screenshot of nascent RNA sequence read distribution along the genome at the FOS locus.

FIG. 3B is a plot showing the number of differentially expressed genes detected using the nascent RNA sequencing protocol versus the steady-state RNA-seq protocol.

FIG. 4 is a plot showing changes in enhancer activity in different cell types in response to diverse stimuli.

FIG. 5 is a plot showing the correlation of the top GWAS variant, rs6964969, with the transcription of the IKZF1 gene.

FIG. 6 is a schematic diagram illustrating an example of the cellular ferroptosis pathway taken from Kathman, S. G. and Cravatt, B. F., Nature Chemical Biology (2020) 16:482-483.

FIG. 7A is a plot showing the transcriptional response of the HT-1080 and IMR90 cell lines to the GPX4 inhibitors ML162 and RSL3.

FIG. 7B is a plot showing the fold induction for HMOX1 mRNA in the HT-1080, A673, and 786-O cell lines exposed to the GPX4 inhibitors ML162 and RSL3.

FIG. 8 is a plot showing the relative expression of HMOX1 in HT-1080 cells in response to different doses of the GPX4 inhibitor RSL3.

FIG. 9 is a plot showing the relative expression of HMOX1 in HT-1080, A673, and 786-O cells in response to different doses of the GPX4 inhibitors RSL3 and ML162 in the presence (light blue bars) or absence ferrostatin-1 (dark blue bars).

FIG. 10 is a plot showing the relative expression of HMOX1 in HT-1080 cells in response to sodium arsenite, hemin, ML162, or RSL3 in the presence (light blue bars) or absence ferrostatin-1 (dark blue bars).

FIG. 11 is a flow diagram illustrating an example of a workflow for screening a potential therapy for its effect on induction of ferroptosis.

FIG. 12 is a flow diagram illustrating an example of a workflow for a method of screening a potential therapy for lipid-peroxide dependent induction of ferroptosis.

FIG. 13 is a flow diagram illustrating an example of a workflow 1300 for a method of identifying a molecular response marker for a biological interaction.

### 6. DETAILED DESCRIPTION OF THE INVENTION

### 6.1. Definitions

“Biologically relevant” means with respect to gene targets of disease-relevant enhancer element variants that the targets have utility or probable utility in the functioning of a biological organism. Biologically relevant includes diagnostically relevant and therapeutically relevant.

“Diagnostically relevant” means with respect to gene targets of disease-relevant enhancer element variants that the targets have utility or probable utility in the screening, diagnosis, monitoring of a disease, monitoring of a disease treatment, or selecting a disease treatment for a subject. For example, individual eRNAs or sets of eRNAs (profiles) may be assayed between disease vs healthy or treated versus untreated groups to identify biomarkers of disease or response to a therapeutic.

“eRNA” means enhancer RNA which represents a class of relatively short non-coding RNA molecules transcribed from the DNA sequence of enhancer regions.

“Misregulation” means a state leading to loss of a normal cell state. The term “misregulate” and “dysregulate” are herein used interchangeably. Examples of misregulation and dysregulation include genetic variation in transcription factors leading to abnormal activation, regulation (activation or repression) or silencing of expression of one or more genes or enhancers.

“Perturbagen” means a substance or condition selected to modulate one or more intracellular processes. Examples include modulation of cell signaling pathways, epigenetic modifications, and/or cancer etiologies. Peturbagens may be small molecules, proteins or peptides, nucleic acids, or other molecules. In various embodiments, a perturbagen may be a drug, hormone, toxin, mutagen, antibody, or a gas, such as oxygen or carbon dioxide. Perturbagens may also be changes in physiochemical conditions of cells, such as temperature, pressure, pH, and lighting conditions. The term “perturbagen” and “stimulus” and “condition” are herein used interchangeably.

“Perturbation” and “perturbate” are used broadly herein to include modulation, modification, stimulation, and mediation. Examples include modulation, modification, stimulation and mediation of cell signaling pathways, epigenetic modifications, cancer etiologies, gene expression signatures, eRNA expression profiles, and transcription factor activity.

“Therapeutically relevant” means with respect to enhancers and gene targets of disease-relevant enhancer element variants that the targets have utility or probable utility in the treatment of a disease or other condition or in enhancement of ordinary biological functioning.

The terms “mutation” and “variant” are herein used interchangeably for purposes of the specification and claims.

### 6.2. Description

various aspects, the invention provides a system and methods of identifying the gene targets of biologically relevant enhancer elements.

The systems and methods of the invention are useful for, among other things:
Identifying functional linkage of chromosomal positions
Linking enhancers to target genes
Linking genomic variants to misregulated target genes
Identifying the gene targets of biologically relevant enhancer elements

In some cases, the system and methods of the invention are useful for identifying the gene targets of therapeutically relevant enhancer element variants. In some cases, the system and methods of the invention are useful for identifying the gene targets of diagnostically relevant enhancer element variants. In one aspect, the invention uses nascent RNA sequencing to identify sites of active transcription events genome-wide, including at all genes and enhancer elements. In one aspect, the invention uses machine learning-based analysis to identify from nascent RNA sequence data active enhancer elements and the genes they regulate. In one aspect, the invention uses nascent RNA sequencing and machine learning-based analysis to identify active enhancer elements and the genes they regulate following experimental stimulation (perturbation) of a cell.

The sequencing data can be viewed as a “histogram” of read depth on the plus (“+”) and minus (“−”) strands of the cellular genome, providing a “snapshot” of which regions of the genome are differentially transcribed in response to the stimulus. A snapshot of transcription events may be captured at a single time point after exposure of a cell to a perturbagen (e.g., a drug) or over a time course after exposure of a cell to a stimulus. A time course of transcription events may be used to reveal a pattern(s) of transcriptional responses to a stimulus, where relationships between the expressions of different genes and enhancers emerge.

In various aspects, the methods of the invention use nascent RNA sequencing and machine learning-based analysis to capture the responses of a certain cell type to a perturbagen and correlate transcription events at an enhancer element to changes in gene transcription, i.e., define the regulatory landscape in response to a perturbagen.

The analyses of data produced by the methods of the invention may be integrated with databases of known genetic variants associated with phenotypic traits or disease and used to generate a linkage map of enhancers and genes that may be dysregulated in a specific disease process.

In one aspect, the methods of the invention make use of perturbagens. In some cases, a perturbagen is selected to directly or indirectly elicit a transcriptional response. In some cases, a perturbagen is selected to have a specific activity and/or target. For example, perturbagens include drugs targeting epigenetic modulators (readers, writers, and erasers of histone modifications), pathway inhibitors (MAPK, JAK/STAT, NFKB), transcription factors (p53, ER, AR, GR, MYC), and components of the proteasomal degradation system such as deubiquitinating enzymes (DUBS).

In some cases, multiple perturbagens are combined to reveal the effect of the combination on transcriptional responses within a cell type.

The perturbagen may be a compound, such as a molecule, protein or peptide, nucleic acid selected to modulate intracellular processes. For example, the perturbagen may be selected from modulators of cell signaling pathways, epigenetic modifications, and/or cancer etiologies. Other examples include modulators targeting, especially those selectively targeting, DNA methyltransferases and histone modifying enzymes.

Table 1 provides examples of epigenetic modulators and targets.

TABLE 1
Epigenetic modulators and targets.

| Compound | Target |
|---|---|
| Panobinostat | Pan-HDAC |
| OTX015 | BRD2/3/4 |
| GSK126 | EZH2 |
| Azacitidine | DNA Methyltransferase |
| WAY-309060 | BRD4 |
| WAY-324030 | CBP/EP300 |
| WAY-6 31668 | DOTIL |
| EBI-2511 | EZH1/2 |
| Droxinostat | HDAC6/8 |
| LMK- 235 | HDAC4/5 |
| (−)-Parthenolide | HDAC, NF-kB, MDM2,p53 |
| WM-1119 | KAT6A |
| CP2 | histone demethylases |
| HLCL-61 HCl | PRMTS |

In some cases, a perturbagen is selected to have a specific epigenetic effect. For example, drugs that affect DNA methylation (methyl transferases, methylases). Table 2 provides examples of pathway modulators and targets.

TABLE 2
Pathway modulators and targets.

| Compound | Target |
|---|---|
| THZI | CDK7 |
| Tozasertib | aurora kinase |
| AG-490 | EGFR |
| Nutlin3a | p53/MDM2 |
| Olaparlb | PARP1/2 |
| Ruxolitinib | JAK |
| Imatinib Mesylate | Abi, c-kit, PDGFR |
| AZD2858 | GSK3 (Wnt activator) |
| TGX221 | PI3K |
| Trametinib | MEK |
| Reversine | adenosine receptor, |
| | aurora kinase |
| AICAR (Acadesine) | AMPK |
| Degrasyn | DUB |
| 2-D08 | sumoylation |
| TNF-alpha | NFKB |
| Forskolin | AC activator |

Further examples of epigenetic modification pathways and their modulators can be found in Bradshaw, R. A., &amp; Dennis, E. A. (2010). Handbook of cell signaling. London: Academic, which is incorporated herein by reference in its entirety.

Exposure to perturbagens may be in vitro or in vivo, e.g., in animal models.

The exposure time of a cell to a perturbagen can be selected to provide a sufficient exposure to yield a “snapshot” of transcription events and characterize the effect of the perturbagen in the cell. The exposure time of a cell to a perturbagen can be selected to reduce non-specific effects caused by exposure to the stimulus, which may reflect general cell stress rather than the direct effect of the stimulus.

In one aspect, a short exposure time period is used. For example, the exposure time may be about 60 minutes or less; or about 30 minutes or less; or about 15 minutes or less; or about 5 minutes or less.

One of skill in the art will understand that the desired exposure time may vary depending on the kind of cell and perturbagen used and the nature of the exposure, e.g., in vitro or in vivo.

A series of exposure times of a cell to a perturbagen can be used to reveal a pattern(s) of transcriptional responses to a perturbagen, where relationships between the expression (transcription levels) of enhancers and/or different genes emerge.

In one aspect, a series of exposure times of a cell to a perturbagen is from about 5 minutes; and about 15 minutes; and about 1 hour; and about 6 hours; and about 24 hours.

In one aspect, the methods of the invention measure nascent RNA in cells. Any biological cells may be used as samples for the methods of the invention. In some embodiments the cells are animal cells. In some embodiments the cells are human cells.

In one aspect of the invention, the cells analyzed are hematopoietic cells. Examples include stem cells, progenitor cells, and/or mature cells of the hematopoietic system. Examples of mature cells include lymphocytes, erythrocytes, megakaryocytes, basophils, mast cells, eosinophils, neutrophils, monocytes, macrophages, Kupffer cells, Langrahans cells, dendritic cells, and osteoblasts.

In one aspect of the invention, the cells analyzed are cancer cells. Examples include acute lymphoblastic leukemia; acute myeloid leukemia; adrenocortical carcinoma; aids-related cancers; aids-related lymphoma; anal cancer; astrocytomas; atypical teratoid/rhabdoid tumor; basal cell carcinoma of the skin; bile duct cancer; bladder cancer; bone cancer (e.g., ewing sarcoma and osteosarcoma and malignant fibrous histiocytoma); brain tumors; breast cancer; bronchial tumors; burkitt lymphoma—see non-hodgkin lymphoma; carcinoid tumor; carcinoma of unknown origin; carcinoma of unknown primary; cardiac tumors; central nervous system cancers; cervical cancer; childhood central nervous system germ cell tumors; childhood extracranial germ cell tumors; childhood rhabdomyosarcoma; childhood vascular tumors; cholangiocarcinoma; chordoma, childhood; chronic lymphocytic leukemia; chronic myelogenous leukemia; chronic myeloproliferative neoplasms; CNS embryonal tumors; colorectal cancer; craniopharyngioma; cutaneous t-cell lymphoma; ductal carcinoma; embryonal tumors; endometrial cancer; ependymoma; esophageal cancer; esthesioneuroblastoma; Ewing sarcoma; extracranial germ cell tumor; extragonadal germ cell tumors; eye cancer; fallopian tube cancer; fibrous histiocytoma of bone; gallbladder cancer; gastric cancer; gastrointestinal carcinoid tumor; gastrointestinal stromal tumors; germ cell tumor; germ cell tumors; gestational trophoblastic disease; hairy cell leukemia; head and neck cancer; heart tumors, childhood; hepatocellular cancer; histiocytosis, Langerhans cell; Hodgkin lymphoma; hypopharyngeal cancer; intraocular melanoma; islet cell tumors, pancreatic neuroendocrine tumors; Kaposi sarcoma; kidney cancer; kidney cancer; Langerhans cell histiocytosis; laryngeal cancer; leukemia; lip and oral cavity cancer; liver cancer; lung cancer; lung cancer (non-small cell, small cell, pleuropulmonary blastoma, and tracheobronchial tumor); lymphoma; male breast cancer; malignant fibrous histiocytoma of bone and osteosarcoma; medulloblastoma; melanoma; melanoma, intraocular; Merkel cell carcinoma; mesothelioma, malignant; metastatic cancer; metastatic squamous neck cancer with occult primary; midline tract carcinoma with nut gene changes; mouth cancer; multiple endocrine neoplasia syndromes; multiple myeloma/plasma cell neoplasms; mycosis fungoides; myelodysplastic syndromes, myelodysplastic/myeloproliferative neoplasms; myelogenous leukemia; myeloid leukemia; myeloproliferative neoplasms, chronic; nasal cavity and paranasal sinus cancer; nasopharyngeal cancer; neuroblastoma; non-Hodgkin lymphoma; non-small cell lung cancer; oral cancer, lip and oral cavity cancer and oropharyngeal cancer; oropharyngeal cancer; osteosarcoma; osteosarcoma and malignant fibrous histiocytoma of bone; ovarian cancer; ovarian germ cell tumors; pancreatic cancer; pancreatic neuroendocrine tumors (islet cell tumors); papillomatosis (childhood laryngeal); paraganglioma; paranasal sinus and nasal cavity cancer; parathyroid cancer; penile cancer; pharyngeal cancer; pheochromocytoma; pituitary tumor; plasma cell neoplasm/multiple myeloma; pleuropulmonary blastoma; pregnancy and breast cancer; primary central nervous system lymphoma; primary CNS lymphoma; primary peritoneal cancer; prostate cancer; rare cancers of childhood; rectal cancer; recurrent cancer; renal cell cancer; retinoblastoma; rhabdomyosarcoma, childhood; salivary gland cancer; sarcoma; Sézary syndrome; skin cancer; small cell lung cancer; small intestine cancer; soft tissue sarcoma; squamous cell carcinoma of the skin—see skin cancer; squamous neck cancer with occult primary, metastatic; stomach cancer; t-cell lymphoma, cutaneous (mycosis fungoides and Sezary syndrome); testicular cancer; throat cancer; thymoma and thymic carcinoma; thyroid cancer; tracheobronchial tumors; transitional cell cancer; transitional cell cancer of the renal pelvis and ureter; ureter and renal pelvis; urethral cancer; uterine cancer, endometrial; uterine sarcoma; vaginal cancer; vascular tumors; vulvar cancer; and Wilms tumor and other childhood kidney tumors.

In one example, the cells tested are tumor cell lines with known mutations.

In another example, cells are tested at different disease stages. For example, a set of cancer cells may be tested that includes multiple cancer stages, such as Phase I, II, III, and/or IV stages, so that response to perturbagens can be determined and compared among states.

In one aspect, a cell sample is a cell line, a xenograft model, or a blood sample.

In one aspect, a cell sample is a cell type selected due to a role it plays in a disease or condition. For example, in one aspect, a cell sample is a human T lymphocyte (“T cell”) or T cell line. T cells are known to play important roles in a number of diseases, including those related to inflammation (Cope, A. P., Arthritis Research & Therapy (2002): S197, which is incorporated herein by reference in its entirety), autoimmunity (Farh, K. K-H., et al., Nature (2015) 518: 337-343, which is incorporated herein by reference in its entirety), and cancer (Corces, M. R., et al., Nature Genetics (2016) 48: 1193-1203, which is incorporated herein by reference in its entirety). It has been demonstrated that disease-associated variants are highly enriched within likely enhancer regions in T cells, which suggests a widespread and important role for enhancer-mediated control of gene expression in this cell type (Tough, D. F. and R. K. Prinjha, Epigenomics (2017) 9(4): 573-584, which is incorporated herein by reference in its entirety). In one aspect, the cell sample is a T cell line such as a culture of Jurkat or CUTTL1 CD4+ T-cells.

In one aspect, two or more different cell samples (e.g., T cell lines) are profiled in parallel to capture changes in transcriptional activity at enhancers and genes in response to a perturbagen(s) in order to find connections likely to be broadly conserved in a population (e.g., human population).

In various embodiments, data produced by the methods of the invention may be combined with other data. For example, data produced by the methods of the invention may be:
mapped to a standard genome;
mapped to or correlated with DNA sequencing data from the cells being analyzed, including, for example, a catalog of GWAS variants;
mapped to or correlated with epigenomic DNA sequencing data from the cells being analyzed;
mapped to or correlated with cell-free DNA or RNA data from a bodily fluid from the subject whose cells are being analyzed;
subject to Hi-C analysis (measures physical proximity of two genomic loci);
subject to Hi-ChIP analysis (measures physical proximity of proteins associated with DNA);
subject to ATAC-seq analysis (measures regions of “open,” or accessible chromatin; and/or
subject to ChIP-seq analysis (measures transcription factor occupancy or histone modifications).

In one aspect, a dataset of known genetic variants is integrated into the analysis to model how non-coding variants (i.e., enhancer variants) could affect distal gene transcription and disease processes.

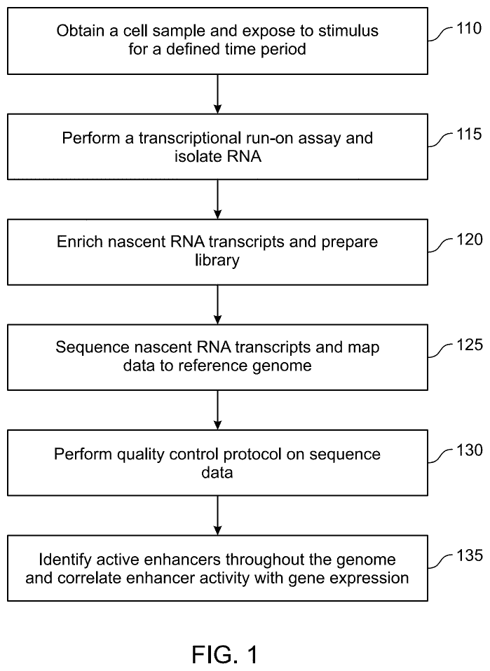

FIG. 1 is a flow diagram illustrating an example of a workflow 100 for measuring changes in transcriptional activity at enhancers and genes throughout the genome of a cell to define the regulatory landscape of the cell in response to a perturbagen. Workflow 100 may include any or all of the following steps as well as additional unspecified steps.

At a step 110, a cell sample is obtained and exposed to a perturbagen for a period of time sufficient to elicit a transcriptional response. For example, a cell sample may be treated with a physiologically relevant dose of a perturbagen for a period of time sufficient to elicit a transcriptional response at enhancers and genes throughout the genome. In one embodiment, the perturbagen is spiked into cell culture media.

In one embodiment, a perturbagen is administered in vivo, and a tissue sample is collected after a period of exposure. For example, a perturbagen may be administered by one or more of the following routes: parenteral (e.g., intravenous, intramuscular, subcutaneous), intraosseous, intrathecal, intraspinal, intracranial, intraperitoneal, intraarticular, intrapleural, intrauterine, intrabladder, intracardiac, oral, ingestion, nasal, ocular, transmucosal (buccal, vaginal, and rectal), and/or transdermal.

In one example, the cell sample is exposed to a perturbagen at physiological temperature (e.g., at about 37° C.) and incubated for about 15 minutes. In another example, the cell sample is exposed to a perturbagen and incubated for about 1 hour. In one example, the cell sample is a T lymphocyte cell line such as a culture of Jurkat or CUTTL1 CD4+ T-cells.

At a step 115, a transcriptional run-on assay is performed.

For example, at the end of the perturbagen incubation period the cell sample is chilled, e.g., to about 4° C. (e.g., placed on ice) to pause active RNA polymerases at the DNA position they were transcribing from (e.g., at a gene or a distal enhancer).

The treated cell sample is permeabilized and washed to remove existing native nucleotide triphosphates (NTPs). To permeabilize, cells may be resuspended in buffer, centrifuged and collected, and resuspended in cell permeabilization buffer.

Examples of suitable permeabilization and wash buffers are provided in Mahat et al., Base-Pair Resolution Genome-Wide Mapping Of Active RNA polymerases using Precision Nuclear Run-On (PRO-seq), Nat Protoc. 2016 August; 11(8): 1455-1476. One of skill in the art will recognize that buffers and conditions for permeabilization and washing may need to be optimized to the specific cell type.

Labeled NTPs are then added to the chilled cell sample. In one example, the labeled NTPs are biotinylated NTPs. Biotin labeled NTPs are commercially available from a variety of sources, e.g., Biotin-11-ATP (PerkinElmer, cat. no. NEL544001EA), Biotin-11-CTP (PerkinElmer, cat. no. NEL542001EA), Biotin-11-GTP (PerkinElmer, cat. no. NEL545001EA), Biotin-11-UTP (PerkinElmer, cat. no. NEL543001EA).

The cell sample is then warmed back to physiological temperature for a period of time sufficient for RNA polymerase to resume transcription and incorporate the biotinylated NTPs into the growing RNA strands (i.e., nascent RNA strands). For example, the harvested cells are warmed back to about 37° C. (36.5-37.5° C.) for about 5 minutes.

Optionally, at the end of the permeabilization cycle, cells may be frozen and stored. For example, following centrifugation, cells may be snap frozen in liquid nitrogen and stored, e.g., at negative 80° C.

Following run-on incubation period, RNA is extracted from the perturbagen-treated cell sample. In one embodiment, extraction makes use of TRIZOL LS Reagent extraction. TRIZOL LS Reagent is commercially available from ThermoFisher Scientific. The TRIZOL LS Reagent User Guide, Pub. No. MAN0000806, Rev. A.0 is incorporated herein by reference.

At a step 120, the nascent labeled RNA transcripts are enriched, and a sequencing library is prepared. In one example, the nascent RNA transcripts have been labeled with biotinylated NTPs as described above, and streptavidin beads are used to capture and isolate the biotinylated nascent RNA transcripts.

At a step 125, the nascent RNA library is sequenced and mapped to a reference genome. The mapped sequencing reads represent the genomic locations where active transcription was occurring at the end of the stimulus incubation period, providing a “snapshot” of transcriptional activity (i.e., RNA polymerase activity) for both enhancers and genes throughout the genome.

At a step 130, quality control protocols may be performed on the sequencing data. In one aspect, a quality control measure is selected to ensure that the enrichment was for nascent RNA, rather than steady state RNA. Enrichment for nascent RNA may be determined based on the distribution of reads across the genome, which differs greatly between a steady state RNA experiment and a nascent RNA experiment. This is because the vast majority of RNA in a cell at steady state exists in its processed form. Mature (processed) mRNA has had its introns excised through splicing, leading to pileup of reads specifically at exons (not introns). However, in nascent RNA sequencing, which measures the position of actively transcribing RNA polymerases, reads are distributed at the positions where RNA polymerase was located at the time of cooling, which includes introns. Therefore, it is expected that the number of exon-mapping reads will be approximately equal to the number of intron mapping reads, when normalized for the total size of the regions. Introns are much longer than exons so it is helpful to normalize the number of reads that map to each location based on the size of the genome intervals that code for introns relative to exons. Typically more than 20 K enhancers and 10 K active promoters are identified in an experiment conducted in the human genome. Sequence data having an exon-intron ratio of <2 and identification of at least 20,000 enhancers and 10,000 promoters per samples will typically be considered to be high quality data. It will be appreciated that different genomes will have different numbers of enhancers and active promoters.

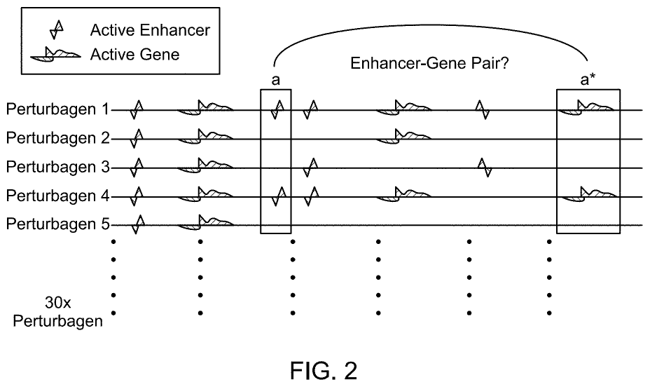

At a step 135, active enhancers throughout the genome are identified and enhancer activity is correlated with gene expression. For example, a machine learning-based model, Tfit, is used to identify active enhancers from the nascent RNA sequence data and correlate enhancer activity with gene transcription (i.e., enhancer-gene linkages are identified). A correlation matrix to link transcriptional activity between enhancers and distal genes using nascent RNA sequence data is described in more detail with reference to FIG. 2.

At a step 140, a dataset of known genetic variants is integrated into the analysis to model how non-coding variants (i.e., enhancer variants) could affect distal gene transcription and disease processes. In one example, a dataset of known single nucleotide polymorphisms (SNPs) identified by Genome Wide Association Studies (GWAS) is integrated into the analysis to determine if identified enhancers overlap known GWAS-identified variants (see, e.g., MacArthur, J., et al., Nucleic Acids Research (2016) 45.D1: D896-D901 and https://www.ebi.ac.uk/gwas/, each of which is incorporated herein by reference in its entirety). Further, while GWAS variants are certainly a rich source of data for this approach, it is also possible for enhancer-linked mutations to be discovered by other techniques. For example, in cancer, most mutations are somatic and do not occur naturally in populations. These mutations could be found by sequencing of cancer cells relative to healthy cells from a patient. Also, in rare disease, enhancer-linked mutations may be discovered by whole genome sequencing of the affected individual and their parents and identifying rare variants.

In one aspect, active enhancers throughout the genome are identified and verified using a machine learning-based model (Azofeifa, J. G, and Dowell, R. D., Bioinformatics (2017) 33(2):227-234, which is incorporated herein by reference in its entirety). For example in one embodiment, Tfit is used to identify enhancers from the nascent RNA sequence data. Based on an exponentially modified Gaussian mixture model, Tfit uses the EM-algorithm to fit highly convolved density functions to non-linearities in nascent transcriptomics data. This method allows for accurate and robust identification of divergent transcription at promoter and enhancer loci alike. This analytical pipeline is capable of identifying transcriptional events at enhancers and coding regions, making putative regulatory connections.

FIG. 2 is a diagram illustrating a correlation matrix for linking transcriptional activity between enhancers and distal genes using nascent RNA sequence data. The basic premise is that the expression of a linked enhancer and gene will change at the same time in response to a perturbagen. In this example, a hypothetical cell is exposed to 30 different perturbagens (i.e., Perturbagen 1, Perturbagen 2, Perturbagen 3, etc.). Exposure of a cell to Perturbagen 1 and Perturbagen 4 elicits a change in the transcription of enhancer “a” and gene “a*” (indicated by boxed region). It has been demonstrated that enhancer transcription levels are directly proportional to their regulatory activity (Mikhaylichenko, O., et al., Genes & Development (2018) 32(1): 42-57; Azofeifa, J. G., et al., Genome Research (2018) 28(3): 334-344; Lizio, M., et al., Genome Biology (2015) 16(1): 22; and Andersson, R., et al., Nature (2014) 507(7493): 455-461, which are incorporated herein by reference in its entirety). Because enhancer transcription is reflective of its current regulatory state, enhancers and genes that correlate in terms of their transcriptional responses to a perturbagen may be linked functionally, i.e., the enhancer element is important for regulating the gene or the enhancer and the gene are regulated by a common regulator.

In one aspect, active enhancer calls may be verified using integration of publicly available chromatin immunoprecipitation sequence data (ChIP-seq data) for matched cell types to compare enhancer profiles. In one example, a H3K27Ac ChIP-seq database is used. For example, publicly available ChIP-seq data for H3K27Ac is available for the Jurkat or CUTTL1 CD4+ T-cell lines (Kloetgen, A., et al., Nature Genetics (2020) 52(4): 388-400, which is incorporated herein by reference in its entirety). H3K27Ac is an epigenetic modification to the DNA packaging protein Histone H3 and is defined as an active enhancer mark.

The significance of each enhancer-gene pair can be assessed, for example, by quantifying the number of reads mapping within 1 kb of each Tfit-identified enhancer, as well as over the length of each gene body. Methods that identify causal linkages through directed acyclic graph permutation can be used to assess the significance of each enhancer gene pair (Pearl, Judea. Causality. (2009) Cambridge University Press, which is incorporated herein by reference in its entirety). The high dimensional enhancer-gene joint distribution can be represented as a Bayesian network where conditional dependencies between enhancers and genes can be easily encoded.

In one aspect, an enhancer-gene connectivity map is generated from the nascent RNA expression data and pattern-matching algorithms are used to suggest or reveal functional connections between the perturbagen, enhancers, and target gene expression.

In one aspect, an enhancer-gene connectivity map is generated from nascent RNA expression data from two or more different cell samples that are profiled in parallel and pattern-matching algorithms are used to suggest or reveal functional connections between perturbagen, enhancer, and target gene expression that may be broadly conserved in human populations.

Predicted enhancer-gene linkages can be validated by disrupting the activity of a disease-relevant enhancer and examining changes in predicted target gene expression in response to the appropriate perturbagen.

In one aspect, CRISPR-based tools are used to disrupt disease-relevant enhancers and the effect of the disruption on expression of predicted target genes is examined using RT-qPCR. For a functional enhancer-gene linkage, disruption of the enhancer will result in altered levels of a predicted target gene in response to exposure to the appropriate perturbagen. For example, an enhancer-gene interaction that is induced under conditions of histone deacetylase (HDAC) inhibition would display a dampened response in a CRISPR-altered cell line. This result would demonstrate that the enhancer is required for proper regulation of its predicted target gene and that mutations altering its activity could affect disease phenotypes through dysregulation of its target gene.

In one aspect, CRISPR-based disruption of disease-relevant enhancers is performed using cultures (e.g., cell lines or primary cultures) of disease relevant cell types. In one example, cultures of Jurkat or CUTTL1 CD4+ T-cells are used to validate predicted enhancer-gene linkages that may be dysregulated in a T cell-mediated disease process. Chi et al., Protocols for CRISPR-mediated genome editing of Jurkat, Biomed Research International 2016:5052369 (2016), which is incorporated herein by reference in its entirety, and CUTLL1 (RRID:CVCL_4966) CD4+ T cell lines have been published. Palomero et al., “CUTLL1, a novel human T-cell lymphoma cell line with t(7;9) rearrangement, aberrant NOTCH1 activation and high sensitivity to gamma-secretase inhibitors,” Leukemia 20:12791287(2006), the entire disclosure of which is incorporated herein by reference.

In one aspect, a CRISPR-mediated non-homologous end joining (NHEJ) strategy that results in deletion of segments of enhancer DNA harboring specific disease-associated variants is used.

In one aspect, a point mutation(s) is introduced into an enhancer region using a CRISPR-mediated genome editing strategy to mimic a specific naturally occurring enhancer mutation (Cong, Le, et al., Science (2013) 339(6121): 819-823, which is incorporated herein by reference in its entirety).

In one aspect, enhancer activity is disrupted using CRISPR inhibition (Yeo, N.C., et al., Nature Methods (2018) 15(8): 611-616, which is incorporated herein by reference in its entirety). For example, a catalytically inactive Cas9 (dCas9) is fused to the KRAB-MeCP2 transcriptional repressor and guided to an enhancer using single guide RNAs (sgRNAs). The recruitment of dCas9-based repressors to enhancers can dramatically reduce enhancer activity as well as transcription of distally regulated genes Thakore, P. I., et al., Nature Methods (2015) 12(12): 1143-1149, which is incorporated herein by reference in its entirety).

Disrupting or silencing the activity of a disease-relevant enhancer (e.g., a GWAS-associated enhancer) will demonstrate whether enhancer activity is functionally linked to the predicted gene, further illuminating the mechanism by which a disease-relevant variant (e.g., a GWAS variant) could exert its function in disease.

In one aspect, the enhancer itself could be a target rather than a gene, e.g., where enhancer activity is modulated through an alteration in chromatin state. Alterations in chromatin state can include, but are not limited to, histone modifications such as methylation or acetylation, and DNA modifications such as methylation. Several examples where enhancer dysregulation has been shown to be necessary and sufficient to drive disease have been reported.

In one aspect, a gene or enhancer identified according to the methods described herein as being dysregulated in disease, is used as a target in a screen for drug candidates having the ability to rescue the “healthy” gene or enhancer activity status. In one embodiment, fluorescent reporters of the target gene/enhancer activity are used to enable high throughput screening of drug candidates. Drug screening can be followed up with a secondary screen using the methods described herein to determine the broader mechanism of action of the drug candidates that rescued the desired gene/enhancer activity.

### 6.3. Examples

### 6.3.1. Sensitivity and Dynamic Range of Nascent RNA Sequencing

To assess the sensitivity and dynamic range of the nascent RNA sequencing assay in gauging a cellular response to a stimulus, the performance of a steady state RNA-seq protocol was compared against a nascent RNA sequencing protocol using a breast cancer cell line (MCF7) model and estrogen treatment to elicit a well characterized, widespread transcriptional response driven by ERα and ERβ transcription factors. Briefly, cell cultures were treated with 100 nM estradiol for 15 minutes or left untreated (i.e., untreated controls) and a steady state RNA-seq protocol or the nascent RNA sequencing assay described herein above was performed.

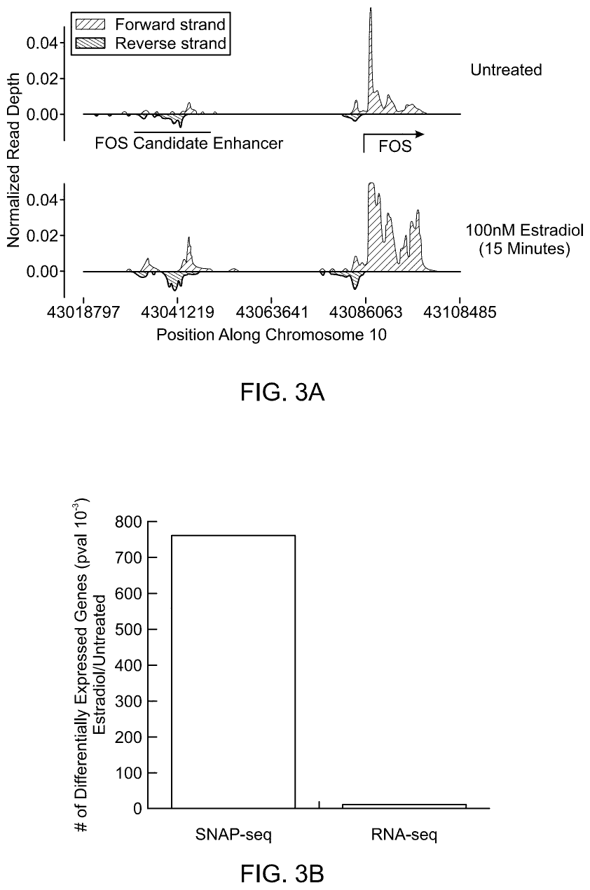

FIG. 3A is a plot showing a “snapshot” of nascent RNA sequence read distribution along the genome at the FOS locus. The data show that within 15 minutes of exposure to 100 nM estradiol, there is a quantitative increase in the level of FOS gene expressions in the treated sample (100 nM estradiol; bottom panel) versus the untreated sample (top panel). The data also show quantitative changes in read distributions at distal candidate FOS gene enhancer elements.

FIG. 3B is plot 410 showing the number of differentially expressed genes detected using the nascent RNA sequencing protocol versus the steady-state RNA-seq protocol. The data show that greater than 700 differentially expressed genes (i.e., estradiol/untreated) were detected using the nascent RNA sequencing protocol (“SNAP-seq”) versus the steady-state RNA-seq protocol (“RNA-seq”). The changes in gene transcription that were identified using the RNA-seq protocol were relatively few, i.e., 8 (pval 10⁻³), and were not directly related to the estrogen signaling pathway. RNA-seq measures steady state RNA, which requires long timeframes to change levels in a statistically significant manner. In the nascent RNA sequencing data (“SNAP-seq”), 762 genes were differentially expressed at the same significance threshold. These were highly enriched for genes linked to the estrogen signaling pathway (pval 10^{-8.6}), highlighting the sensitivity of the nascent RNA sequencing (“SNAP-seq”) assay and its ability to define the correct biological mechanism affected by a stimulus.

### 6.3.2. Detecting Changes in Enhancer/Gene Activity Among Cell Types in Response to Different Stimuli

To demonstrate that nascent RNA sequencing and associated data analysis can be used to detect changes in enhancer/gene activity among different cell types in response to different stimuli, the effect of more than 100 different stimuli on enhancer activity was profiled in more than 40 cell types.

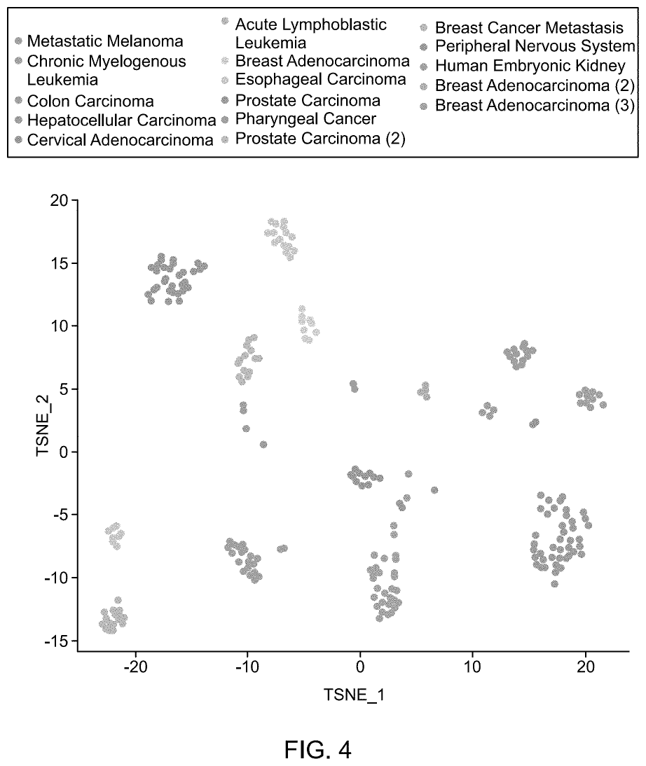

FIG. 4 is a plot showing changes in enhancer activity in different cell types in response to diverse stimuli. The plot is a TSNE plot where each dot represents the activity of greater than 20,000 enhancers from a single biological replicate of a nascent RNA sequencing (“SNAP-seq”) sample (n=450). Samples cluster strongly by cell type. The data show that the nascent RNA sequencing (“SNAP-seq”) assay distinguishes cell types.

To demonstrate that the locations of active enhancers can be used to investigate how genetic variants could influence distal gene expression, a database of more than 900 nascent RNA sequencing (“SNAP-seq”) experiments using dozens of cell types was examined and the level of active transcription near every non-genic, GWAS-identified variant was correlated with the transcription of each gene located more than 10 kb away from the variant.

Table 3 shows the top 20 enhancer-gene linkages revealed with Pearson correlation and p-values.

TABLE 3
Top 20 enhancer-gene linkages with
Pearson correlation and p-values.

| | Predicted Target | Correlation | |
|---|---|---|---|
| GWAS rs ID | Gene | (Pearson’s) | pvalue (−log10) |
| rs6964969 | IKZF1 | 0.926728 | 149.703908 |
| rs10466905 | LTBR | 0.925601 | 148.59161 |
| rs751979 | SALL1 | 0.923483 | 146.546651 |
| rs1227734 | GDF15 | 0.916681 | 140.357747 |
| rs28364580 | AXL | 0.914062 | 138.11447 |
| rs7204669 | PRSS22 | 0.906219 | 131.803163 |
| rs11665748 | KLK4 | 0.905071 | 130.926335 |
| rs3956705 | RP9P | 0.904308 | 130.34963 |
| rs11665748 | KLK3 | 0.901736 | 128.440529 |
| rs140032935 | ABO | 0.897925 | 125.707163 |
| rs4385425 | CD1E | 0.892516 | 122.007493 |
| rs2479409 | PCSK9 | 0.89157 | 121.380917 |
| rs140824606 | CBLC | 0.890687 | 120.801617 |
| rs915125 | FAM46A | 0.884398 | 116.811669 |
| rs6964969 | RCSD1 | 0.880782 | 114.621125 |
| rs28364580 | SERPINE1 | 0.876553 | 112.14801 |
| rs140824606 | MARVELD3 | 0.876177 | 111.932914 |
| rs780432508 | LITAF | 0.8756 | 111.603458 |
| rs7248710 | FXYD5 | 0.873202 | 110.252232 |
| rs6964969 | SPN | 0.872465 | 109.842269 |

The analysis identified dozens of statistically significant correlations (Pearson's correlation >0.926), including one variant “rs6964969” whose local transcription is extremely highly correlated with transcription of the IKZF1 gene. The rs6964969 variant has been associated with childhood acute lymphoblastic leukemia.

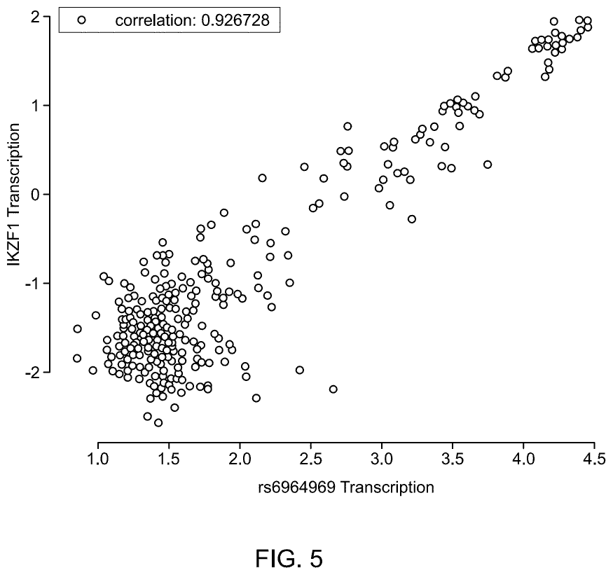

FIG. 5 is a plot showing the correlation of the top GWAS variant, rs6964969, with the transcription of the IKZF1 gene. The data suggests that the rs6964969 variant may function by altering the transcription of the IKZF1 gene.

In one aspect, a method is provided for identifying markers of biological interactions.

### 6.3.3. Identifying Biomarkers of Ferroptosis

Ferroptosis is a recently described form of regulated cell death driven by the iron-dependent accumulation of lipid peroxides (Dixon, S. J., et al., Cell (2012) 149(5): 10601072, which is incorporated herein by reference in its entirety). Ferroptosis has been implicated in a wide variety of disease settings, including kidney injury and cancer, as well as cardiovascular, neurodegenerative, and hepatic diseases (Han, C., et al., Frontiers in Pharmacology (2020) 11: 239, which is incorporated herein by reference in its entirety). Therefore, there is a need for quantifiable biomarkers of ferroptosis in order to design and develop strategies for modulation of this important phenotype.

In healthy cells, ferroptosis is prevented mainly by the activity of a single protein, GPX4. GPX4 is a peroxidase that reduces lipid peroxides to inert lipid alcohols. When GPX4 is disabled, lipid peroxides rapidly accumulate, leading to cell death in certain cell types (Yang, W. S., et al., Proceedings of the National Academy of Sciences (2016) 113(34): E4966-E4975, which is incorporated herein by reference in its entirety). Inhibition of GPX4 using, for example, chemical inhibitors is a promising strategy for induction of ferroptosis in cancer cells (Kathman, S. G. and Cravatt, B. F., Nature Chemical Biology (2020) 16:482-483, which is incorporated herein by reference in its entirety).

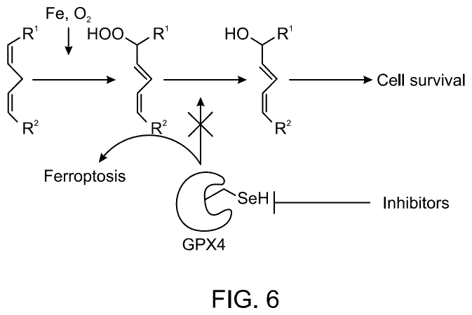

FIG. 6 is a schematic diagram illustrating an example of the cellular ferroptosis pathway. In the presence of oxidative stress (e.g., iron (Fe) and molecular oxygen (O2)), polyunsaturated fatty acids are converted to lipid peroxides. The accumulation of lipid peroxide induces ferroptosis. GPX4 is an enzyme in a cellular defense pathway against ferroptosis. GPX4 reduces lipid peroxides into inert lipid alcohols which are nontoxic. Inhibition of GPX4 causes accumulation of lipid peroxides and cell death. The Figure is adapted from Kathman and Cravatt (2020).

### 6.3.3.1 HMOX1 Induction as a Ferroptosis Sensor

To assess the transcriptional response of a cell to inhibition of GPX4, we exposed two cell lines to two different chemical inhibitors of GPX4 (ML162 and RSL3; available from Sigma Aldrich and Apex Bio respectively) and examined the transcriptional responses using a nascent RNA sequencing protocol. Briefly, a fibrosarcoma cell line HT-1080 and a lung fibroblast cell line IMR90 were exposed to 1 μM ML162 or 1 μM RSL3 for 1 hour and the nascent RNA sequencing protocol was performed.

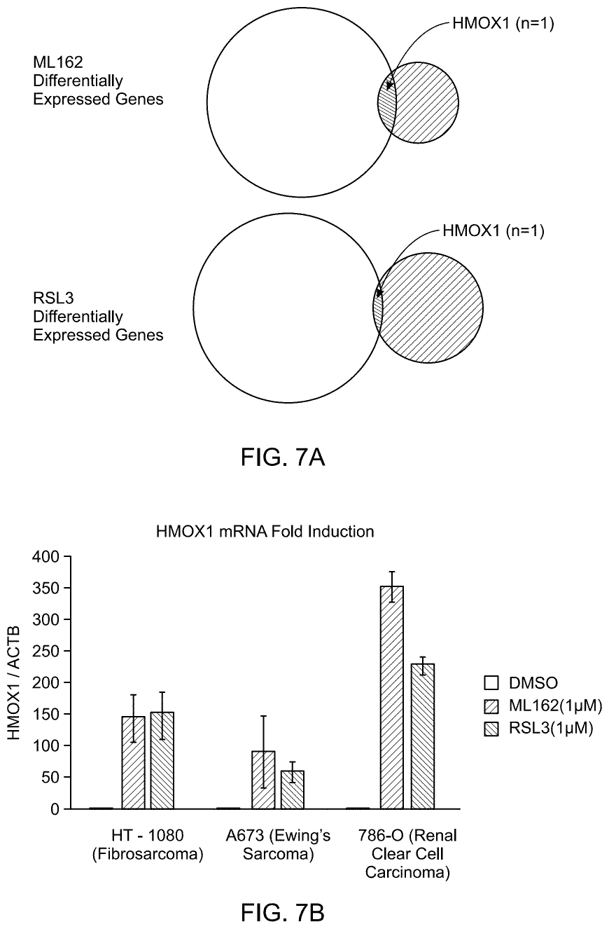

FIG. 7A is a plot showing the transcriptional response of the HT-1080 and IMR90 cell lines to the GPX4 inhibitors ML162 and RSL3. The data show that the number of genes with significant changes in gene transcription was greater in the HT-1080 cell line relative to the IMR90 cell line for both inhibitors. A single gene, HMOX1 (heme oxygenase 1) was strongly differentially expressed in response to both inhibitors in both HT-1080 (Pval<10⁻²) and IMR90 (Pval 10⁻³) cell lines. Expression of HMOX1 was strongly and specifically induced by both inhibitors, which suggests that HMOX1 may be used as a “molecular response biomarker” or sensor for GPX4 inhibition and thus lipid peroxidation and ferroptosis.

To confirm HMOX1 induction by the GPX4 inhibitors, an RT-qPCR assay was performed using three ferroptosis sensitive cancer cell lines: the fibrosarcoma cell line HT-1080, a Ewing's sarcoma cell line A673, and a renal clear cell carcinoma cell line 786-O. Briefly, cells were exposed to 1 μM ML162 or 1 μM RSL3 for 4 hours, RNA was isolated and an RT-qPCR assay was performed.

FIG. 7B is a plot showing the fold induction for HMOX1 mRNA in the HT-1080, A673, and 786-O cell lines exposed to the GPX4 inhibitors ML162 and RSL3. Data were normalized to a DMSO control for each cell line (n=2 biological replicates). The data show HMOX1 is induced by the GPX4 inhibitors ML162 and RSL3 in all three cancer cell types. The data confirms that HMOX1 may be used as a sensor for ferroptosis.

To further validate induction of HMOX1 mRNA expression by GPX4 inhibition, RT-qPCR was performed using varying doses of the RSL3 inhibitor. Briefly, HT-1080 cells were exposed to 5 μM RSL3, 1 μM RSL3, and 0.2 μM RSL3 for four hours, RNA was isolated and an RT-qPCR assay was performed to quantify HMOX1 expression. DMSO was used as a control exposure.

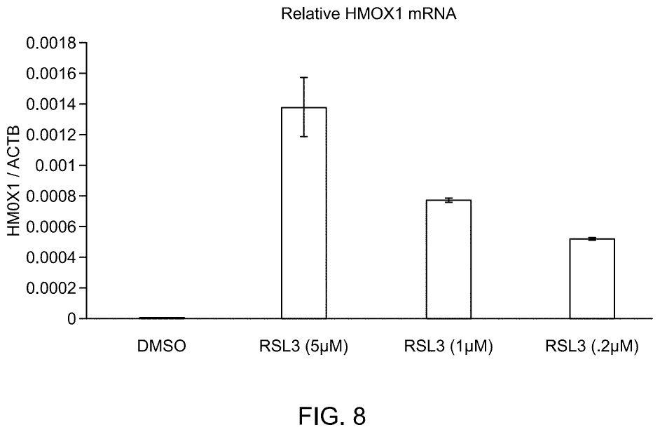

FIG. 8 is a plot showing the relative expression level of HMOX1 in HT-1080 cells in response to different doses of the GPX4 inhibitor RSL3. The data is plotted as the expression of HMOX1 relative to expression of the ACTB gene. The data show that induction of HMOX1 gene expression is proportional to the dose of the GPX4 inhibitor, which suggests that HMOX1 induction represents a quantitative readout on the amount of GPX4 inhibition and thus ferroptosis.

Elevated levels of HMOX1 have been observed in conditions of general oxidative stress (Poss K. D. and Tonegawa, S. Proceedings of the National Academy of Sciences (1997) 94(2): 10925-10930; and Takada, T., et al., Arthritis Research & Therapy (2015) 17(1): 285, which are incorporated herein by reference in its entirety). Ferrostatin-1, which is capable of reducing lipid peroxides, has been used as a tool to determine whether cell death is being driven specifically by lipid peroxidation as opposed to other forms of oxidative stress (Miotto, G., et al., Redox Biology (2020) 28: 101328, which is incorporated herein by reference in its entirety). For example, when cells are treated with small molecules that induce lipid peroxidation (e.g., GPX4 inhibitors), addition of ferrostatin-1 to the culture media rescues cellular viability. This ferroptosis-protective effect has been observed in the HT-1080, A673, and 786-O cells lines (Dixon, S. J., et al., Cell (2012) 149(5): 1060-1072; and Li, A., et al., JCI Insight (2017) 2(7): e90777, which are incorporated herein by reference in its entirety).

Ferrostatin-1 acts in an analogous manner to GPX4 in that when ferrostatin-1 is added to cells in the presence of a GPX4 inhibitor, ferrostatin-1 is able to complement or replace the activity of GPX4.

To determine whether GPX4 inhibitor-mediated HMOX1 induction is dependent on lipid peroxidation, an experiment was designed to determine whether addition of ferrostatin-1 to the culture media of HT-1080, A673, and 786-O cells was capable of rescuing the HMOX1 induction that was observed upon GPX4 inhibition. Briefly, HT-1080, A673, and 786-O cells were exposed to 1 μM ML162 or 1 μM RLS3 with or without 1 μM ferrostatin-1 for four hours, RNA was isolated and an RT-qPCR assay was performed to quantify HMOX1 expression. DMSO was used as a control exposure.

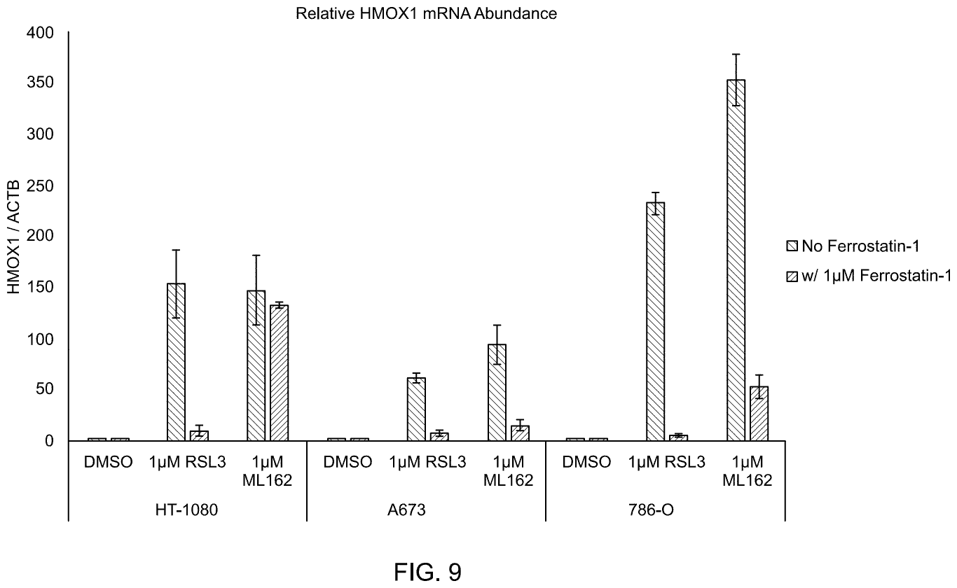

FIG. 9 is a plot showing the relative expression of HMOX1 in HT-1080, A673, and 786-O cells in response to different doses of the GPX4 inhibitors RSL3 and ML162 in the presence (light blue bars) or absence ferrostatin-1 (dark blue bars). The data is plotted as the expression of HMOX1 relative to expression of the ACTB gene. The data show that HMOX1 expression was observed in all three cell lines by both RSL3 and ML162 treatments, confirming that HMOX1 is induced upon GPX4 inhibition in multiple cellular contexts. The data also show that the addition of ferrostatin-1 to the culture media was sufficient to prevent induction of HMOX1 in most conditions, suggesting that HMOX1 induction is generally being driven specifically by the accumulation of lipid peroxides and not by generalized oxidative stress. In the HT-1080 cell line, ML162 induced HMOX1 even in the presence of ferrostatin-1. This observation suggests that ML162-mediated HMOX1 induction is not due solely to lipid peroxidation, but may be due to an off target effect of ML162 (e.g., general oxidative stress) in some cell types.

To test whether HMOX1 induction in response to other forms of oxidative stress is dependent on lipid peroxidation, HT-1080 cells were exposed to either sodium arsenite or hemin. Sodium arsenite and hemin are known to be inducers of general oxidative stress in cells. Briefly, HT-1080 cells were exposed to sodium arsenite (80 μM), hemin (5 μM), ML162 (1 μM), or RSL3 (1 μM) with or without 1 μM ferrostatin-1 for four hours, RNA was isolated and an RT-qPCR assay was performed to quantify HMOX1 expression. DMSO was used as a control exposure.

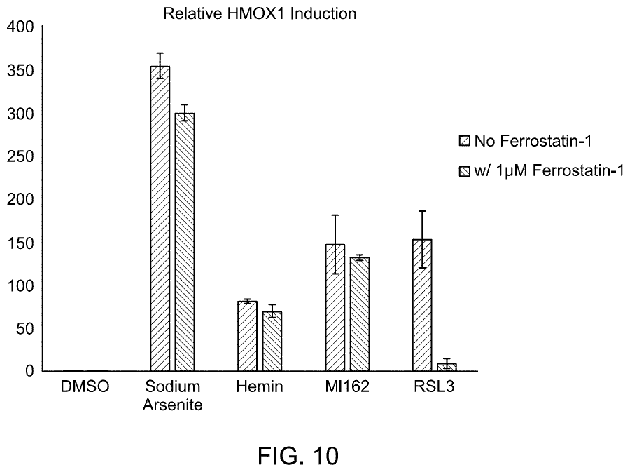

FIG. 10 is a plot showing the relative expression of HMOX1 in HT-1080 cells in response to sodium arsenite, hemin, ML162, or RSL3 in the presence (light blue bars) or absence ferrostatin-1 (dark blue bars). Data were normalized to a DMSO control. The data show that sodium arsenite- and hemin-mediated induction of HMOX1 was not affected by the presence of ferrostatin-1, which suggests that while HMOX1 induction is induced during ferroptosis, its induction in the presence of ferrostatin-1 is indicative of generalized oxidative stress, rather than canonical ferroptosis. The data also show that RSL3-mediated induction of HMOX1 is dependent on lipid peroxidation (i.e., addition of ferrostatin-1 to the culture media was sufficient to prevent induction of HMOX1).

These observations suggest that drug candidates designed to induce ferroptosis (e.g., GPX4 inhibitors) can be screened for their ability to induce HMOX1 expression in the absence of ferrostatin-1, but to a lesser extent in the presence of ferrostatin-1. This is because these drug candidates are likely to be causing ferroptosis without stimulating much generalized oxidative stress.

### 6.3.4. Additional Methods

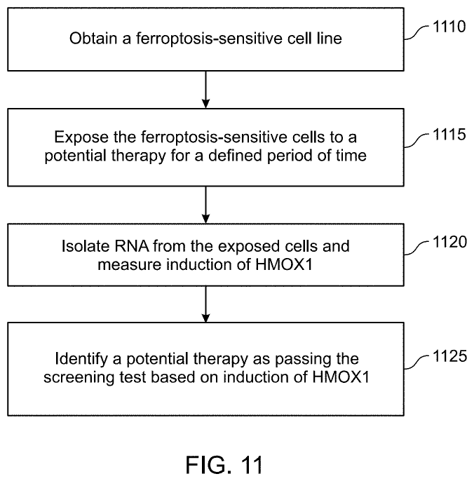

In one embodiment of the invention, a method is provided of screening a potential therapy for its effect on induction of ferroptosis. FIG. 11 is a flow diagram illustrating an example of a workflow 1100 for screening a potential therapy for its effect on induction of ferroptosis using HMOX1 as a molecular marker of ferroptosis. Workflow 1100 may include any or all of the following steps as well as additional unspecified steps.

At a step 1110, a ferroptosis-sensitive cell line is obtained. In one example, the ferroptosis-sensitive cell sample may be a cancer cell line.

At a step 1115, the ferroptosis-sensitive cell line is exposed to a potential therapy, or a library of potential therapies, for a defined period of time. For example, the ferroptosis-sensitive cell line may be exposed to the potential therapy for a period of time sufficient to elicit a transcriptional response. In one example, the potential therapy may be a potential inhibitor of GPX4 function.

At a step 1120, RNA is isolated from the exposed cells and induction of HMOX1 is measured. In one example, RT-qPCR may be used to measure induction of HMOX1 gene expression.

At a step 1125, identify a potential therapy as passing the screening test based on induction of HMOX1. For example, a potential therapy is identified as passing the screening test if it induces increased expression of HMOX1. The degree of HMOX1 induction can be used to generate a score for the potential therapy.

In some embodiments, the method of the invention further comprises measuring cell death in response to exposure to a potential therapy. In one example, cell death may be measured by a Cell Titer Glo assay, which measures ATP production. The measure of cell death can be used in combination with the degree of induction of HMOX1 to score a potential therapy.

In one aspect, the invention provides a method of screening a potential therapy for lipid-peroxide dependent induction of ferroptosis.

In one aspect, the method of the invention makes use of a lipophilic antioxidant in combination with a potential therapy to distinguish the induction of lipid peroxide-induced ferroptosis from generalized oxidative stress.

In one aspect, the screening strategy uses induction of HMOX1 as a molecular response marker for lipid peroxide-dependent induction of ferroptosis, wherein induction of HMOX1 in the absence of the lipophilic antioxidant but not in the presence of the lipophilic antioxidant indicates ferroptosis without generalized oxidative stress.

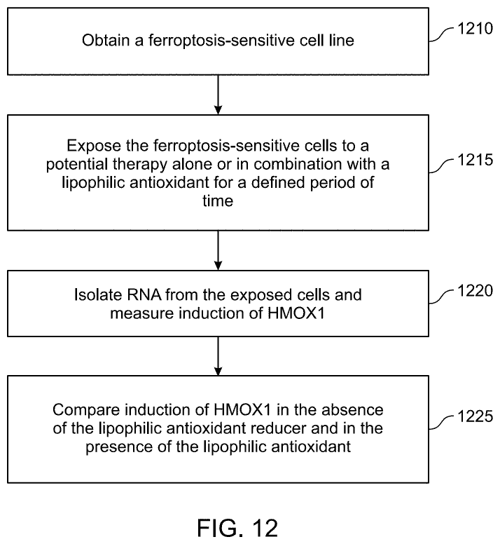

FIG. 12 is a flow diagram illustrating an example of a workflow 1200 for a method of screening a potential therapy for lipid-peroxide dependent induction of ferroptosis. Workflow 1200 may include any or all of the following steps as well as additional unspecified steps.

At a step 1210, a ferroptosis-sensitive cell line is obtained. In one example, the ferroptosis-sensitive cell sample may be a cancer cell line.

At a step 1215, the ferroptosis-sensitive cell line is exposed to a potential therapy alone or in combination with a lipophilic antioxidant for a defined period of time. For example, the ferroptosis-sensitive cell line may be exposed to the potential therapy alone or in combination with a lipophilic antioxidant for a period of time sufficient to elicit a transcriptional response. In one example, the potential therapy is a potential inhibitor of GPX4 function. In one example, the lipophilic antioxidant is a ferrostatin, such as ferrostatin-1 or an active modified version of ferrostatin-1.

At a step 1220, RNA is isolated from the exposed cells and induction of HMOX1 is measured. In one example, RT-qPCR may be used to measure induction of HMOX1 gene expression.

At a step 1125, the induction of HMOX1 in the absence of the lipophilic antioxidant and in the presence of the lipophilic antioxidant are compared. Induction of HMOX1 in the absence of the lipophilic antioxidant but not in the presence of the lipophilic antioxidant indicates ferroptosis without generalized oxidative stress (i.e., lipid peroxide-dependent ferroptosis).

In some embodiments of the invention, the screening strategy uses cell death as an indicator of lipid-peroxide dependent induction of ferroptosis, wherein cell death occurs in the absence of the lipophilic antioxidant but not in the presence of the lipophilic antioxidant.

In one aspect, the invention provides a method of identifying a “molecular response” marker or markers for a biological interaction. In various aspects, the method uses quantifying RNA expression in a cell line exposed to a perturbagen, wherein the perturbagen is selected to induce the biological interaction. The identification of a marker and/or set of markers for the biological interaction may be determined based on the pattern of RNA expression in the absence of the perturbagen relative to the pattern of RNA expression in the presence of the perturbagen. In one embodiment, the RNA expression is quantified by nascent-RNA sequencing.

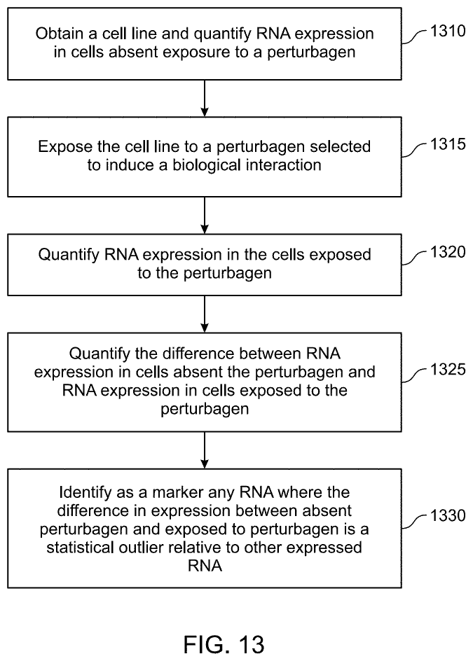

FIG. 13 is a flow diagram illustrating an example of a workflow 1300 for a method of identifying a molecular response marker for a biological interaction. Workflow 1300 may include any or all of the following steps as well as additional unspecified steps.

At a step 1310, a cell line is obtained and RNA expression in cells absent a perturbagen is quantified.

At a step 1315, the cell line is exposed to a perturbagen selected to induce a biological interaction.

At a step 1320, RNA expression in the cells exposed to the perturbagen is quantified. The RNA expression can be quantified by nascent-RNA sequencing. The measurement may in some cases be taken immediately following exposure to the perturbagen.

At a step 1325, the difference between RNA expression in cells absent the perturbagen and RNA expression in cells exposed to the perturbagen is quantified.

At a step 1330, an RNA marker is identified as any RNA wherein the difference in expression between absent perturbagen and exposed to perturbagen is a statistical outlier relative to other expressed RNAs. As discussed below in more detail, at least some steps of these methods can be implemented using a processor executing software stored in a tangible, non-transitory storage medium. For example, the software can be stored in the long-term memory (e.g., solid state memory) in a genetic sequencer, executed by the processor in the genetic sequencer. In other embodiments, the software can be stored in a separate system configured to access sequencing information from a genetic sequencer.

### 6.3.5. Concluding Remarks

While the disclosed subject matter is amenable to various modifications and alternative forms, specific embodiments are described herein in detail. The intention, however, is not to limit the disclosure to the particular embodiments described. On the contrary, the disclosure is intended to cover all modifications, equivalents, and alternatives falling within the scope of the disclosure as defined by the appended claims.

Similarly, although illustrative methods may be described herein, the description of the methods should not be interpreted as implying any requirement of, or particular order among or between, the various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As the terms are used herein with respect to ranges, “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

We claim:

1. A method of screening a therapy for induction of ferroptosis, the method comprising:
a. exposing a cell line to a potential therapy;
b. measuring induction of Heme Oxygenase 1 (HMOX1) resulting from the exposing the cell line to the potential therapy; and
c. identifying the potential therapy as passing or failing the screening of the therapy for induction of ferroptosis based on the measuring of (b).

2. The method of claim 1, wherein the cell line comprises a ferroptosis-sensitive cell line or a ferroptosis-resistant cell line.

3. The method of claim 2, wherein the cell line comprises a ferroptosis-sensitive cell line, and the ferroptosis-sensitive cell line comprises a cancer cell line.

4. The method of claim 1, wherein the cell line comprises cancer cells.

5. The method of claim 1, wherein the potential therapy comprises a drug therapy.

6. The method of claim 1, wherein the potential therapy comprises a potential inhibitor of Glutathione Peroxidase 4 (GPX4).

7. The method of claim 6, wherein the potential inhibitor of GPX4 comprises a small molecule, a protein, or a peptide.

8. The method of claim 6, wherein the potential inhibitor of GPX4 comprises a gene therapy that modulates GPX4 function.

9. The method of claim 1, wherein the measuring induction of HMOX1 comprises quantifying a level of HMOX1 gene expression in the cell line.

10. The method of claim 9, wherein the quantifying the level of the HMOX1 gene expression comprises measuring:
i. an increase in the HMOX1 gene expression in the exposed cell line relative to a control;
ii. no substantial change in the HMOX1 gene expression in the exposed cell line relative to the control; or
iii. a reduction in the HMOX1 gene expression in the exposed cell line relative to the control.

11. The method of claim 1, wherein the measuring induction of HMOX1 comprises measuring protein expression of HMOX1.

12. The method of claim 1, wherein the measuring induction of HMOX1 comprises measuring a change in HMOX1 mRNA abundance.

13. The method of claim 1, wherein the measuring induction of HMOX1 comprises quantifying a level of HMOX1 gene transcription using a nascent transcription assay.

14. The method of claim 1, wherein the measuring induction of HMOX1 comprises using RT-qPCR to quantify HMOX1 expression in the cell line.

15. The method of claim 1, wherein the potential therapy is identified as passing the screening if the measuring induction of HMOX1 shows in an increased expression of HMOX1 relative to a control.

16. The method of claim 1, wherein the potential therapy is identified as passing the screening if the measuring induction of HMOX1 shows an increased expression of HMOX1 of at least 1.5× relative to a control.

17. The method of claim 1, wherein the potential therapy is identified as passing the screening if the measuring induction of HMOX1 shows in a lack of change or a reduced expression of HMOX1 relative to a control.

18. The method of claim 1, further comprising generating a score for the potential therapy based on a degree of the induction of HMOX1.

19. The method of claim 18, wherein the generating the score for the potential therapy is performed without directly quantifying accumulation of lipid peroxides.

20. The method of claim 1, further comprising measuring cell death of cells in the cell line in response to the exposing of the cells in the cell line to the potential therapy in (a).

21. The method of claim 20, wherein the measuring the cell death comprises using a live/dead assay.

22. The method of claim 20, further comprising using a degree of the cell death in combination with a degree of induction of HMOX1 as measured in (b) to generate a score for the potential therapy.

23. The method of claim 1, wherein the cell line comprises a ferroptosis-sensitive cell line, the method further comprising:
i. exposing the ferroptosis-sensitive cell line to the potential therapy in the presence of a lipophilic antioxidant;
ii. measuring induction of HMOX1 resulting from the exposing of the ferroptosis-sensitive cell line to the potential therapy in the presence of the lipophilic antioxidant;
iii. measuring induction of HMOX1 resulting from the exposing of the ferroptosis-sensitive cell line to the potential therapy in the absence of the lipophilic antioxidant; and
iv. comparing the measured induction of HMOX1 in (ii) and the measured induction of HMOX1 in (iii),
wherein the induction of HMOX1 in (iii), but not in (ii), indicates ferroptosis without generalized oxidative stress, and
wherein the induction of HMOX1 in both (ii) and (iii) indicates ferroptosis with generalized oxidative stress.

24. The method of claim 23, wherein the potential therapy passes the screening when the potential therapy induces HMOX1 in the absence, but not in the presence, of the lipophilic antioxidant.

25. The method of claim 23, wherein the lipophilic antioxidant comprises a ferrostatin.

26. The method of claim 23, wherein the lipophilic antioxidant comprises liproxstatin.

27. The method of claim 23, wherein the lipophilic antioxidant comprises an iron chelator.

28. The method of claim 23, further comprising measuring cell death of the ferroptosis-sensitive cell line.

29. The method of claim 24, further comprising administering the potential therapy to a subject if the potential therapy passes the screening.

30. The method of claim 29, wherein the potential therapy induces expression of HMOX1 in the absence, but not in the presence, of the lipophilic antioxidant.
